(19) United States
(12) Patent Application Publication
(10) Pub. No.: US 2011/0144117 A1
(43) Pub. Date: Jun. 16, 2011

(54) Pharmaceutical Dosage Form Containing Tetrahydrobiopterin

(75) Inventors: Rudolf Widmann, Purkersdorf (AT); Andreas Bernkop-Schnuerch, St. Veit/Glan (AT)

(73) Assignee: OrPha Swiss GmbH, Kusnacht (CH)

(21) Appl. No.: 13/058,625

(22) PCT Filed: Aug. 7, 2009
(86) PCT No.: PCT/AT09/00306

§ 371 Date: Feb. 11, 2011

(30) Foreign Application Priority Data
Aug. 12, 2008 (AT) A1254/2008

(51) Int. Cl. A61K 31/4985; A61P 3/00

(52) U.S. Cl. 514/249

(57) ABSTRACT

Pharmaceutical dosage form comprising tetrahydrobiopterin, or a metabolic precursor thereof, comprising an aqueous solution of tetrahydrobiopterin, or a metabolic precursor thereof, and an antioxidant in a dispensing container and a dosing means for the dosed dispensation of the aqueous solution, as well as the use of an antioxidant for stabilizing an aqueous solution of tetrahydrobiopterin, or a metabolic precursor thereof, for a storage period of more than at least one month.

### BACKGROUND OF THE INVENTION

### Field of the Invention

The present invention relates to a pharmaceutical dosage form containing tetrahydrobiopterin, which may be used for treating phenylketonuria variants caused by the deficiency of tetrahydrobiopterin.

Tetrahydrobiopterin constitutes a cofactor of the phenylalanine hydroxylase (PAH) and also a cofactor of the alkyl glycerol monooxygenase, the tyrosine hydroxylase (TH), the tryptophane hydroxylase (TPH) and the nitric oxide hydroxylases, where it was initially identified. Therefore, tetrahydrobiopterin is necessary for the conversion of phenylalanine to tyrosine and for the production of the monoamine neurotransmitters dopamine, epinephrine, norepinephrine and serotonin (Miwa et al., Arch. Biochem. Biophys. 1985; 239:234-241).

Hyperphenylalaninemia is a common inherited metabolic disease, which in most cases (approximately 98%) develops because of a phenylalanine hydroxylase deficiency due to mutations in the phenylalanine hydroxylase gene (Scriver et al. Hum Mutat 2000; 15: 99-104). The associated phenotypes range in severity from classic phenylketonuria (>1200 μmol/l phenylalanine in blood plasma) to mild phenylketonuria (600-1200 μmol/l phenylalanine in blood plasma) and mild hyperphenylalaninemia (120-600 μmol/l phenylalanine in blood plasma). Patients with both classic and mild phenylketonuria require lifelong dietary protein restriction to prevent neurological sequelae and to ensure normal cognitive development.

Studies have shown that dietary supplementation of tetrahydrobiopterin reduces phenylalanine blood plasma concentration in patients exhibiting mutations in the PAH gene (Kure et al. J Pediatr 1999; 135:375-8). However, only certain mutations in the PAH gene appeared to be indicative of responsiveness to tetrahydrobiopterin, wherein other mutations of PAH were not. The tetrahydrobiopterin-sensitive hyperphenylalaninemia can be further differentiated into a tetrahydrobiopterin-sensitive PAH deficiency and a defect in the synthesis or regeneration of tetrahydrobiopterin, i.e. typical (severe) forms of GTP cyclohydrolase 1 (GTPCH), dihydropteridine reductase (DHPR) and 6-pyruvoyl-tetrahydropterin synthase (PTPS) deficiencies.

Approximately 1-3% of patients with persistent hyperphenylalaninemia have a deficiency in tetrahydrobiopterin synthesis (Danks et al. J. Inherited Metab. Dis. 1978; 1: 49-53; Berlow et al. Pediatrics. 1980; 65:837-839). The majority of patients with defects in synthesis of tetrahydrobiopterin frequently exhibit mutations in the gene coding for the phosphate-eliminating enzyme PTPS, which catalyzes the second step of the biosynthesis of tetrahydrobiopterin from dihydroneopterin triphosphate. Such patients suffering from tetrahydrobiopterin-sensitive hyperphenylalaninemia due to a deficient tetrahydrobiopterin pathway, which can be accompanied by neurological degeneration, are unresponsive to low-phenylalanine dietary treatment.

Therefore, treatment regimens, which include the daily oral administration of tetrahydrobiopterin, are necessary (Muntau et al., New England Journal of Medicine 2002; 347: 2122-2132). Alternatively, it might also be conceivable to administer L-sepiapterin. Niederwieser at al. reported that the symptoms of hyperphenylalaninemia transiently disappeared in a patient with a dihydrobiopterin deficiency upon administration of a single dose of L-sepiapterin (Niederwieser et al. Euro J Pediatr 1982; 138(2):110-2). Accordingly, Nichol and co-workers presented a salvage pathway responsible for the regeneration of tetrahydrobiopterin from dihydrobiopterin, via a dioxo compound and L-sepiapterin in kidney and liver, which is catalyzed by DHPR (Nichol et al., Proc. Natl. Acad. Sci. 1983; Vol. 83: 1546-1550). Such recycling of tetrahydrobiopterin appears to be absolutely essential to ensure a continuous supply of reduced tetrahydrobiopterin and to prevent accumulation of harmful metabolites. In fact, several mutations in the DHPR gene are known to culminate into malignant hyperphenylalaninemia (Danks et al. J. Inherit. Metab. Dis. 1978; 1:49-53).

However, administration of tetrahydrobiopterin as of yet is accompanied by several problems. Solid inert moisture-free formulations are thus far known as the only option for long-term storage of tetrahydrobiopterin, since tetrahydrobiopterin is quickly degraded in the presence of an aqueous solvent. As a consequence, the individual dosing, which is directly linked to the body weight of the respective patient, varies widely from newborns to adults and has to be achieved with a solid formulation of tetrahydrobiopterin, e.g. powder or tablet. This is complex and difficult, because, on the one hand, each individual powder dose has to be weighed separately and, on the other hand, once tetrahydrobiopterin powder is exposed to oxygen, e.g. because the sealing of the package is broken, the drug is rapidly oxidized. Consequently, it has not been possible to provide tetrahydrobiopterin as bulk powder formulation for individual weighing by the patient. All attempts to solve this problem are focusing on strategies to improve the stability of tetrahydrobiopterin in a dry solid formulation and to dissolve sealed single unit solid formulations immediately before administration.

Current strategies to overcome this pharmaceutical and technological challenges are focusing on more stable derivatives of tetrahydrobiopterin, for example, more stable crystalline solid forms (see, e.g. WO2005/065018), sealing under nitrogen in ampoules and storage at ≦−20° C. (Schricks Laboratories, Switzerland) as well as the addition of antioxidants to solid formulations (Fiege et al., Mol Genet Metab. 2004 January; 81(1):45-51). Making use of all these strategies, however, provides stability of multi-unit delivery systems only for a couple of weeks unless stored at −20° C. or below. Thus, a suitable formulation for a simplified individualized oral treatment with tetrahydrobiopterin is needed.

Dosage forms of tetrahydrobiopterin, or a metabolic precursor thereof, are, for example, known from EP 0 209 689, EP 0 906 913, JP 10 338 637, or JP 63 267 781.

There has surprisingly been found a pharmaceutical dosage form for the oral administration of tetrahydrobiopterin, or a metabolic precursor thereof, by means of which it is easy to dose tetrahydrobiopterin, or a metabolic precursor thereof, in accordance with the weight of the individual patient and which is suitable to be administered to newborns, children or adults and which comprises tetrahydrobiopterin, or a metabolic precursor thereof, in a stable form.

### SUMMARY OF THE INVENTION

In one aspect the present invention provides a pharmaceutical dosage form comprising tetrahydrobiopterin, or a metabolic precursor thereof, characterized in that it comprises an aqueous solution of tetrahydrobiopterin, or a metabolic precursor thereof, and of an antioxidant in a dispensing container and that it comprises a dosage means for the dosed dispensation of the aqueous solution.

A pharmaceutical dosage form, which is provided according to the present invention, is herein also designated as “(pharmaceutical) dosage form according to (in accordance with) the present invention”.

A pharmaceutical dosage form according to the present invention comprises an oral dosage form for the liquid administration of tetrahydrobiopterin, or a metabolic precursor thereof, in the form of an aqueous solution.

A pharmaceutical dosage form according to the present invention comprises tetrahydrobiopterin, or a metabolic precursor thereof, and an antioxidant, which are dissolved in an aqueous solvent, and optionally one or several pharmaceutically acceptable auxiliary agents.

The aqueous solvent in a dosage form according to the present invention is a solvent which allows for the protonization of salts or acids dissolved therein and comprises water or water in compound with another hydrophilic solvent, e.g. ethanol, isopropanol, polyethylene glycol, glycerol, dimethyl sulfoxide as well as combinations thereof.

Preferably, an aqueous solution in a dosage form according to the present invention comprises water as a solvent component, preferably at least 50%, at least 60%, at least 70%, at least 80%, at least 90%, at least 99% water; and most preferably the solvent is pure water (100%). In case that the metabolic precursor of tetrahydrobiopterin used has a lower solubility in water than e.g. L-sepiapterin, which has a solubility of 0.17 g/100 g H₂O in pure water, it is preferred that mixtures of water with another hydrophilic solvent are used to improve solubility, e.g. propylene glycol or glycerine. It is also possible to improve solubility by various methods known to improve solubility including e.g. ultrasonification.

Tetrahydrobiopterin (5,6,7,8-tetrahydro-biopterin) in a dosage form according to the present invention is a compound of the formula

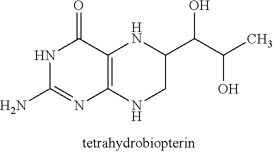

corresponding to the reduced form of biopterin. The chemical reduction of biopterin yields two diastereoisomers, this is (6S)- and (6R)-5,6,7,8-tetrahydro-L-biopterin, wherein (6R)-5,6,7,8-tetrahydro-L-biopterin corresponds to the natural form of tetrahydrobiopterin. Tetrahydrobiopterin is also known under the name sapropterin or BH₄. (6R)-5,6,7,8-tetrahydro-L-biopterin in the form of a dihydrochloride salt is commercially available under the trade name Kuvan®. (6R,S)-5,6,7,8-tetrahydro-L-biopterin in a ratio of (6R)/(6S)=70/30 is, for example, available in the form of a dihydrochloride from Schircks Laboratories, Switzerland.

A metabolic precursor of tetrahydrobiopterin in a dosage form according to the present invention includes a pterin or biopterin intermediate compound, which may be converted/reduced on the intracellular level direct into 5,6,7,8-tetrahydrobiopterin or another pterin or biopterin intermediate compound, either via the de novo-path for the biological synthesis of tetrahydrobiopterin or via the salvage pathway responsible for the regeneration of tetrahydrobiopterin. A metabolic precursor of the (natural) tetrahydrobiopterin, this is L-sepiapterin of the formula

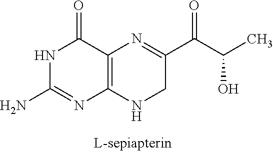

(CAS 17094-01-8) may be, upon administration via the pterin salvage pathway, which is dependent on the sepiapterin reductase activity (˜7,8-dihydro-biopterin:NADP⁺-oxidoreductase), on the intracellular level converted into (6R)-5,6,7,8-tetrahydro-L-biopterin (Blau et al., Mol. Genet. Metab. 2001; 74(1-2):172-185). The metabolic precursor of the tetrahydrobiopterin may be present in the form of a salt, such as, e.g., that of (6S)- and (6R)-5,6,7,8-tetrahydro-L-biopterin.

### DESCRIPTION OF THE FIGURES

FIG. 1. Influence of N-acetyl cysteine used in indicated concentrations (m/v) on the stability of tetrahydrobiopterin in an aqueous solution. Indicated values are means of at least 3 experiments±SD.

FIG. 2. Influence of cysteine used in indicated concentrations (m/v) on the stability of tetrahydrobiopterin in an aqueous solution. Indicated values are means of at least 3 experiments±SD.

### DETAILED DESCRIPTION OF THE INVENTION

In a dosage form according to the present invention tetrahydrobiopterin is preferably selected from the group consisting of (6R)-5,6,7,8-tetrahydro-L-biopterin and (6R,S)-5,6,7,8-tetrahydro-L-biopterin, e.g. in the ratio of (6R)/(6S)=70:30, and their salts, in particular acid addition salts, e.g. hydrochloride salts such as dihydrochloride salts; especially preferred is (6R)-5,6,7,8-tetrahydro-L-biopterin, preferably in the form of a salt such as dihydrochloride salt. A metabolic precursor of tetrahydrobiopterin according to the present invention is preferably L-sepiapterin, e.g. in the form of a salt.

An antioxidant in a dosage form according to the present invention comprises pharmaceutically acceptable sulfhydryl compounds, this is compounds having one or several sulfhydryl groups, for example sulfhydryl groups that may be oxidized into covalent, bridge forming disulfide compounds. Preferred examples of such compounds comprise amino acids or amino acid derivatives having sulfhydryl groups, this is sulfhydryl compounds, in particular cysteine compounds, for example comprising cysteine, e.g. L-cysteine and compounds derived therefrom, such as, e.g., N-acetyl cysteine, homocysteine, N-acetyl homocysteine, cysteine methyl ester, cysteine ethyl ester, homocysteine methyl ester, and homocysteine ethyl ester; or mixtures of two or more different sulfhydryl compounds, optionally in the form of a salt. A preferred antioxidant in a dosage form according to the present invention is cysteine, such as L-cysteine, or N-acetyl cysteine, e.g. in the form of a salt such a hydrochloride.

The molar ratio of tetrahydrobiopterin, or a metabolic precursor thereof, and an antioxidant in a dosage form according to the present invention comprises a ratio in the range of 1.5:1 to 1:4 or more, e.g. 1.1:1; 1:1; 1:1.1; 1:1.2; 1:1.3; 1:1.4; 1:1.5; 1:2; 1:3 or 1:4; or more; preferably the molar ratio is at least 1:1, such as, e.g., 1:1 to 1:4, e.g. 1:1 to 1:1.5, preferably about 1:1. The upper limit of the antioxidants contained in the dosage form according to the present invention is determined by the solubility of the respective antioxidant in the respective aqueous solvent used.

Stabilization of the tetrahydrobiopterin, or a metabolic precursor thereof, in a dosage form according to the present invention may already be observed with a molar ratio of 1.5:1 (tetrahydrobiopterin:antioxidant), a significant stabilization with a molar ratio of (about) 1:1.

In the case of (6R)-5,6,7,8-tetrahydro-L-biopterin in the form of a dichloride as tetrahydrobiopterin and L-cysteine in the form of a hydrochloride as antioxidant there is preferably used 1 g antioxidant for 1.5 g to 2 g, e.g. for 1.6 g, 1.7 g, 1.8 g, 1.9 g or 2.0 g tetrahydrobiopterin, e.g. 1 g antioxidant for 2.0 g tetrahydrobiopterin.

The preferred concentration of tetrahydrobiopterin and/or a precursor thereof in a dosage form of the present invention should allow the administration of sufficient tetrahydrobiopterin and/or a metabolic precursor thereof. This amount of liquid will comprise sufficient tetrahydrobiopterin and/or a metabolic precursor thereof to trigger a therapeutic response in a patient suffering from tetrahydrobiopterin-sensitive hyperphenylalaninemia. In a specific embodiment of the present invention the exact dosage is determined by monitoring the efficiency in order to alleviate the symptoms of tetrahydrobiopterin-sensitive hyperphenylalaninemia, which are noticeable e.g. by normal physiological levels of phenylalanine and normal production of catecholamines and serotonin in patients with a tetrahydrobiopterin synthesis deficiency. Typically, the required dose will be in the range of between 5 mg and 80 mg of tetrahydrobiopterin and/or a metabolic precursor thereof per kg body weight, preferably in the range between 10 mg to 40 mg per kg body weight. Said dose may be administered in a single dose or multiple smaller doses administered over the course of the day, in particular in two doses of 2.5 mg to 40 mg tetrahydrobiopterin and/or a metabolic precursor thereof per kg body weight, preferably in the range between 5 mg to 20 mg per kg body weight for each dose. It is particularly preferred that 10 mg/kg body weight are administered twice a day and, thus, for a newborn of e.g. 4 kg, it will be required that about 40 mg of tetrahydrobiopterin are comprised within a given amount of the liquid formulation, while for a 100 kg adult about 1000 mg of tetrahydrobiopterin have to be comprised in a given amount of the liquid formulation.

A dosage form according to the present invention comprises tetrahydrobiopterin, or a metabolic precursor thereof, in particular (6R)-5,6,7,8-tetrahydro-L-biopterin.2HCl and/or a metabolic precursor thereof, in the range of 0.5 wt % to 50.0 wt %, preferably 2.0 wt % to 25 wt. %, e.g. 2.0 wt % to 20 wt. %, more preferably of 4.0 wt % to 15 wt %, e.g. 5.0 wt % to 15 wt %, e.g. about 10 wt %. The concentration of the antioxidant in a dosage form of the present invention is adjusted to provide a sufficient molar ratio in order to stabilize the tetrahydrobiopterin.

The aqueous solution in a dosage form according to the present invention preferably has an acidic pH, e.g. an acidic pH below 5, preferably below 3, and more preferably below 2, such as 5.0, 4.5, 4.0, 3.5, 3.0, 2.5, 2.0, 1.5, 1.0 or less, e.g. 1.0 t 5.5. Typically, the desired “acidity” is achieved by simply dissolving tetrahydrobiopterin acid addition salts, e.g. (6R)-5,6,7,8-tetrahydro-L-biopterin dihydrochloride acid addition salt. If the desired low pHs are not achieved by the respective tetrahydrobiopterin, it is preferred to adjust the desired pH value with an acidifying substance. The desired acidic pH below 2 of an acidic liquid formulation containing L-sepiapterin, which is available in a 99% pure crystalline form (provided from Cayman Chemical), may be adjusted, for example, by supplementing an appropriate amount of an acidifying substance.

A pharmaceutical dosage form according to the present invention may further comprise one or several pharmaceutically acceptable auxiliary agents. Pharmaceutically acceptable auxiliary agents, which may be present in a dosage form of the present invention, preferably comprise auxiliary agents used in galenics, flavor additives, preservatives, acidifying substances and/or coloring agents.

A pharmaceutical dosage form according to the present invention may be obtained through the addition of an aqueous solvent to a solid pharmaceutical composition, comprising tetrahydrobiopterin, or a metabolic precursor thereof, an antioxidant, and optionally pharmaceutically acceptable auxiliary agents, or through the preparation of an aqueous solution of tetrahydrobiopterin, or a metabolic precursor thereof, and an antioxidant, optionally by adding a pharmaceutically acceptable auxiliary agent, wherein the aqueous solution is introduced into/in a dispensing container which can be connected with a dosage means for the dosed dispensation of the aqueous solution, wherein the water content of a solid pharmaceutical composition is preferably in the range of 1.0% to 5.0%, e.g. 1% to 4.0%.

Such a solid pharmaceutical composition comprises, for example, powder, pellets or granulates such as tablets, in which there are present, apart from flavor additives, preservatives, acidifying substances and/or coloring agents as pharmaceutically acceptable auxiliary agents, further pharmaceutically acceptable auxiliary agents such as desiccants, fillers and/or granulating agents. If such solid pharmaceutical compositions are used for the preparation of a dosage form according to the present invention, the aqueous solution in a dosage form of the present invention may also contain such further pharmaceutically acceptable auxiliary agents.

A flavor additive according to the present invention comprises a compound, which may help to improve the taste of a pharmaceutical formulation according to the present invention, such as, for example, naturally occurring sweeteners, artificial nutritive sweeteners, flavoring agents, which may, for example, cause a cinnamon, a vanillin, a cherry, a strawberry, an orange, a banana and/or an apple flavor.

A naturally occurring sweetener used in a dosage form of the present invention comprises monosaccharides such as glucose, galactose, mannose, xylitol; disaccharides such as fructose, sucrose, lactose, maltose, mixtures of mono- and/or disaccharides with polysaccharides such as maltodextrin; an artificial sweetener comprises sugar substitutes like sorbit, mannit, aspartame (e.g. Nutrasweet™ and Equal™), saccharine and acesulfame K.

A sweetener which may be present in a dosage form according to the present invention preferably comprises glucose, galactose, mannose, xylitol, fructose, saccharose, lactose, maltose and/or maltodextrin.

A preservative present in a dosage form of the present invention comprises all natural and synthetic compounds that are pharmaceutically authorized and can be added to foodstuff, beverages or pharmaceuticals, in order to prolong the period of time before these will be spoilt, either by microbial growth or due to undesired chemical changes, such as antimicrobial compounds prohibiting growth of bacteria and fungi, e.g. benzoates, borates, sorbates, carbonates, acetates such as sodium benzoate, potassium benzoate, benzoic acid, sodium sorbate, potassium sorbate, sorbic acid, p-hydroxybenzoic acid methyl ester and p-hydroxybenzoic acid ethyl ester, sodium borates, dimethyl dicarbonate or dimethyl acetate. Other preservatives may additionally exhibit an anti-oxidizing effect and may contribute to the stabilization of tetrahydrobiopterin in a dosage form according to the present invention. Preservatives having an anti-oxidizing effect comprise e.g. phenolic compounds such as butylated hydroxyanisole (BHA) and the related compound butylated hydroxytoluene (BHT).

A preservative in a dosage form according to the present invention preferably comprises sodium benzoate, sodium borate, dimethyl dicarbonate, dimethyl acetate, butylated hydroxyanisole or butylated hydroxytoluene.

An acidifying substance provided according to the present invention comprises pharmaceutically acceptable, salt forming acids, see e.g. Berge et al., Pharmaceutical salts 1977 J. Pharm. Sci.; 66(1):1-19, e.g. solid acids. The stability of a dosage form according to the present invention might be further enhanced, if an acidifying substance of the present invention also exhibit an anti-oxidizing property. Examples of such acidifying substances include citric acid, tartaric acid, and benzoic acid.

An aqueous solution containing tetrahydrobiopterin and/or a metabolic precursor thereof can sometimes change its color if stored for a prolonged period of time; such discoloration, however, does not have to implicitly mean that it has lost its therapeutic activity, Thus, a dosage form of the present invention may include a coloring agent in order to maintain the external appearance of the aqueous solution.

A coloring agent, which may be provided in a dosage form of the present invention, includes pharmaceutically acceptable natural or artificially synthesized dyes. A great variety of such pharmaceutically acceptable dyes have been known to be suitable for use in pharmaceutical compositions, for example natural dyes such as annatto extract, anthocyanins, β-carotene, beta APO 8, carotenal, black currant, burnt sugar, canthaxanthin, caramel, carbo medicinalis, carmine, carmine blue, carminic acid, carrot, chlorophyll, chlorophyllin, cochineal extract, copper-chlorophyll, copper-chlorophyllin, curcumin, curcumin/CU-chloro, elderberry, grape, hibiscus, lutein, mixed carotenoids, paprika, riboflavin, spinach, stinging nettle, titanium dioxide, turmeric, natural colors, aronia/redfruit, beet juice colors, paprika extract, paprika oleoresin; or artificial dyes such as allura red, amaranth, carmoisine, fast red E, erythrosine, green S, patent blue V, ponceau 4R, quinoline yellow, red 2G, sunset yellow, and tartrazine.

If there is used a solid pharmaceutical composition, comprising tetrahydrobiopterin, or a metabolic precursor thereof, for the preparation of a pharmaceutical dosage form according to the present invention, the dosage form according to the present invention may also include desiccants, fillers and/or granulating agents, apart from the ingredients already mentioned above.

A desiccant provided in a dosage form of the present invention comprises a substance, which is capable of compensating for the hygroscopic property of a pharmaceutical agent or additional excipients; e.g. SiO₂, present e.g. in Syloid® AL or aerosols, or CaCO₃.

A filler, which may be provided in a dosage form of the present invention, e.g. comprises lactose, saccharose, cellulose and their derivatives (e.g. hydroxy ethyl cellulose, and hydroxy propyl cellulose), starch, and talcum and/or calcium hydrogen phosphate.

A granulating agent, which may be provided in a dosage form according to the present invention, comprises, e.g., ethanol, gelatine, crospovidone and/or polyvinyl pyrrolidone.

A dispensing container in a dosage form of the present invention includes an air and liquid tight container, preferably made from glass, e.g. brown glass material or plastic, e.g. an optically opaque plastic material. The dispensing container preferably has a capacity of 10 ml to 1000 ml, such as 25 ml to 1000 ml, e.g. 25 ml to 500 ml, such as 50 ml to 200 ml, e.g. 10 ml, 25 ml, 50 ml, 75 ml, 100 ml, 125 ml, 150 ml, 175 ml, 200 ml, 300 ml, 400 ml, 500 ml, 600 ml, 700 ml, 800 ml, 900 ml or 1000 ml.

The dispensing container in a dosage form of the present invention may include connecting means, e.g. screw threads, for connecting the dosing means that is suitable for the dosed dispensation of the aqueous solution with or at the dispensing container.

A dosing means for the dosed dispensation of the aqueous solution in a dosage form of the present invention includes a liquid dosing means, for example a pump, syringe or pipette pump, suitable for dispensing the desired amounts of the aqueous solution.

The dosing means preferably includes an actuator, which may be arranged in the dispensing container or may be extending into the dispensing container. The dosing means preferably includes a pumping device as an actuator, which can be movably mounted on the dispensing container and extends into the dispensing container, e.g. via a cannula. By moving the pumping device towards the outlet of the dispensing container a well-dosed quantity of the aqueous solution emerges from the outlet of the dispensing container.

The dosing means is suitable for dispensing aliquots of the aqueous solution, for exampling of 100 μl to 2000 μl, such as 200 μl to 1500 μl e.g. 500 μl to 1000 μl; e.g. 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl or 2000 μl. The exact volume of the aliquot of the aqueous solution dispensed by the dosing means may be either predetermined or mechanically adjustable to the desired volume.

In one embodiment the present invention provides a dosage form according to the present invention, wherein the dosing means is firmly connected with the dispensing container.

The dispensing container with or without dosing means may in addition be sealed in a film consisting of aluminum composite in order to prevent oxidation of the substance before use.

In another embodiment, the present invention provides a kit comprising the aqueous solution in a dispensing container and the dosing means separated from each other, however, in one single package.

An aqueous solution in a dosage form according to the present invention may be administered to a patient in a suitable dose with the aid of a dosing means which is connected or will be connected with the dispensing container. Administration may also be performed via direct oral ingestion by the patient, or the suitable dose may be added, as close in time to consumption as possible, to a foodstuff such as milk, yoghurt, alcohol-fee beverages, soft drinks, e.g. orange or apple juice, Coca Cola, soup, water, baby food, for ingestion by the patient. There has been found out, for example, that dilutions of an aqueous solution in a dosage form according to the present invention with breast-milk or dilutions of an aqueous solution in a dosage form according to the present invention with infant formula, for example a 12.2 wt % solution of an infant formula such as Aptamil®, HA Pre®, Milupae, will not coagulate; for example, if there is used a ratio of 1 part of an aqueous solution in a dosage form according to the present invention, containing for example 10% tetrahydrobiopterin, with 5 parts of breast-milk or with 5 parts of a dilution of an aqueous dosage form with a solution of an infant formula, for example a 12.2 wt % solution of an infant formula such as Aptamil®, HA Pre®, Milupa®. There may also be administered an aqueous solution in a dosage form according to the present invention hence also in combination with breast-milk or with a solution of an infant formula such as Aptamil®, HA Pre®, Milupa®, or also with commercially available baby food, to children and infants.

In one aspect a dosage form according to the present invention may be prepared by preparing an aqueous solution of tetrahydrobiopterin, or a metabolic precursor thereof, and an antioxidant, optionally with addition of excipients, wherein the aqueous solution is introduced into/in a dispensing container connected with the dosing means for the dosed dispensation of the aqueous solution.

The method of preparation for a dosage form according to the present invention comprises in one aspect the following steps:

(i) weighing of the antioxidant, e.g. a sulfhydryl compound and tetrahydrobiopterin, or a metabolic precursor thereof, and optionally pharmaceutically acceptable auxiliary agents,
(ii) optionally mixing of the ingredients of (i),
(iii) dissolving the antioxidant, the tetrahydrobiopterin, or a metabolic precursor thereof, and optionally pharmaceutically acceptable auxiliary agents or mixtures thereof in an aqueous solvent, and
(iv) optionally introducing the aqueous solution or the aqueous solutions of (iii) into a dispensing container which is connected or will be connected with the dosing means for the dosed dispensation of the aqueous solution.

Dissolution of the antioxidant, tetrahydrobiopterin, or a metabolic precursor thereof, and optionally pharmaceutically acceptable auxiliary agents may be carried out either by separately dissolving the ingredients in an aqueous solvent, or one or several, optionally all, ingredients are together dissolved in an aqueous solvent.

Introducing the aqueous solution or the aqueous solutions into a dispensing container, which is connected or will be connected with a dosing means for the dosed dispensation of the aqueous solution, may be realized by filling an aqueous solution or aqueous solutions of one, several or all ingredients into the dispensing container, or an aqueous solution of one, several or all ingredients is directly prepared in the dispensing container, and optionally aqueous solutions of one or several ingredients are further added.

In another aspect the preparation of a dosage form according to the present invention may be obtained from a solid dosage form, e.g. in the form of powders, pellets, granulates, tablets, of tetrahydrobiopterin, or a metabolic precursor thereof, and an antioxidant, and optionally pharmaceutically acceptable auxiliary agents, via addition of an aqueous solvent, wherein the aqueous solution is introduced into a dispensing container which is connected or will be connected with a dosage means for the dosed dispensation of the aqueous solution.

Such a solid dosage form may, for example, be provided in a vessel, preferably with air-tight sealing.

If the solid dispensation form is e.g. a powder, a method of preparation will comprise the following steps:

(i) weighing an antioxidant, e.g. a sulfhydryl compound, and tetrahydrobiopterin, or a metabolic precursor thereof, and, optionally pharmaceutically acceptable auxiliary agents,
(ii) mixing the ingredients of (i),
(iii) dissolving the mixture of (ii) in an aqueous solvent, optionally in a dispensing container attached at a dosing means for the dosed dispensation of the aqueous solution, and
(iv) optionally introducing the aqueous solution of (iii) into a dispensing container, which is connected or will be connected with a dosing means for the dosed dispensation of the aqueous solution.

If the solid dosage form is provided e.g. in an air-tight container, e.g. in sachets, in the form of a granulate, a method for preparation will comprise, for example, the following steps:

(i) weighing of pharmaceutically acceptable auxiliary agents, an antioxidant, e.g. a sulfhydryl compound and of tetrahydrobiopterin, or a metabolic precursor thereof,
(ii) mixing of the ingredients of (i),
(iii) granulating the mixture of (ii) with a granulating agent,
(iv) pressure or heat drying of the granulates of (iii),
(v) optionally and preferably sizing the granulates of (iv), e.g. by sieving,
(vi) optionally adding and admixing of further pharmaceutically acceptable auxiliary agents to granulates of (v) and
(vii) dissolving the mixture of (vi) in an aqueous solvent, optionally in a dispensing container attached at a dosing means for the dosed dispensation of the aqueous solution, and
(viii) optionally introducing the aqueous solution of (vii) into a dispensing container which is connected or will be connected with a dosing means for the dosed dispensation of the aqueous solution.

If the solid composition is provided in the form of e.g., pellets, a method of preparation will comprise the following steps:

(i) weighing of pharmaceutically acceptable auxiliary agents, an antioxidant, e.g. a sulfhydryl compound and of tetrahydrobiopterin and/or a metabolic precursor thereof,
(ii) mixing of the ingredients of (i),
(iii) granulating the mixture of (ii) with a granulating agent,
(iv) pelletizing the granulates of (iii),
(v) drying the granulates of (iv),
(vi) dissolving the mixture of (v) in an aqueous solvent, optionally in a dispensing container attached at a dosing means for the dosed dispensation of the aqueous solution, and
(vii) optionally introducing the aqueous solution of (vi) into a dispensing container which is connected or will be connected with a dosing means for the dosed dispensation of the aqueous solution.

Powders, pellets or granulates of a solid composition may be pressed optionally, for example by use of suitable pharmaceutically acceptable auxiliary agents, into pellets, e.g. tablets.

If the solid composition is provided for example in the form of pellets, e.g. tablets, a method of preparation will comprise the following steps:

(i) dissolving the pellets, e.g. tablets, in an aqueous solvent, optionally in a dispensing container attached at a dosing means for the dosed dispensation of the aqueous solution, and
(ii) optionally introducing the aqueous solution of (i) into a dispensing container, which is connected or will be connected with a dosing means for the dosed dispensation of the aqueous solution.

It has been shown that an aqueous solution in a dosage form according to the present invention is stable at a temperature of 2° C. to 60° C., e.g. 4° C. to 40° C., e.g. at room temperature.

An aqueous solution in a dosage form according to the invention is designated herein as “stable”, if it contains at least 95%, e.g. 96%, 97%, 98% or 99% of the initial concentration of tetrahydrobiopterin, or a metabolic precursor thereof, in particular (6R)-5,6,7,8-tetrahydro-L-biopterin.2HCl, upon storage of at least one month, e.g. of 2, 3, 4, 5, 6, 9 or 12 months. The initial concentration of tetrahydrobiopterin, or a metabolic precursor thereof, is defined as the concentration that will be determined immediately after the provision of the aqueous solution in a dosage form according to the present invention by use of suitable means such as, e.g. HPLC.

Possibly as a consequence of a high dosing precision, a dosage form of the present invention is in particular suitable for the administration of tetrahydrobiopterin, or of a metabolic precursor thereof, to infants or children, wherein e.g. the aqueous solution in a dosage form according to the present invention is administered advantageously in combination with a foodstuff, e.g. in combination with milk such as breast-milk and milk on the basis of infant formula, alcohol-free beverages, soups, baby food.

In another aspect the present invention provides a pharmaceutical dosage form, which is in particular suitable for the administration of tetrahydrobiopterin, or a metabolic precursor thereof, to infants or children, for example the use of a pharmaceutical dosage form according to the present invention for the preparation of a medicament for treating phenylketonuria variants caused by the deficiency of tetrahydrobiopterin, in particular hyperphenylalaninemia, by means of administration of tetrahydrobiopterin, or a metabolic precursor thereof, to children or infants, in particular by administration in combination with milk or baby food.

In another aspect the present invention provides, the use of an antioxidant, e.g. the use of a sulfhydryl compound, for the stabilization of an aqueous solution of tetrahydrobiopterin, or a metabolic precursor thereof, for a storage period, e.g. for storage in open state, of at least one month, e.g. of 2, 3, 4, 5, 6, 9 or 12 months, wherein in particular the molar ratio of the tetrahydrobiopterin, or the metabolic precursor thereof, and the antioxidant comprises a ratio of about 1.5:1 to 1:4, in particular of 1:1 to 1:1.5.

Another aspect the present invention provides the use of an antioxidant, in particular a sulfhydryl compound, in particular a cysteine, for stabilizing an aqueous solution of tetrahydrobiopterin, or a metabolic precursor thereof, wherein the tetrahydrobiopterin, or the metabolic precursor thereof, are stable at 40° C.±2° C. and 75%±5% relative humidity and/or at 25° C.±2° C. at 60%±5% relative humidity, in particular wherein 95%, in particular 96%, 97%, 97.5%, 98% or 99%, of the initial concentration of the tetrahydrobiopterin, or a metabolic precursor thereof, will be maintained upon storage of at least one month, in particular 2, 3, 4, 5, 6, 9 or 12 months.

An aqueous solution in a dosage form according to the present invention may be used as a medicament or for the production of a medicament for treating, ameliorating or curing a disease requiring the administration of tetrahydrobiopterin and/or a metabolic precursor thereof, such as e.g. tetrahydrobiopterin-sensitive hyperphenylalaninemia, diabetes type II, several forms of hypertension, erectile dysfunction and subsets of disorders with altered neurotransmitter metabolism, such as Parkinson's disease.

The term “hyperphenylalaninemia” as used herein encompasses a metabolic disorder in a mammal, preferably a human being, characterized by increased blood serum levels of phenylalanine, typically in the range of between 120 and 600 mmol/1.

The term “tetrahydrobiopterin-sensitive hyperphenylalaninemia”, as used herein, refers to the variant of hyperphenylalaninemia, defined by a phenylalanine hydroxylase deficiency or a defect in the synthesis of tetrahydrobiopterin, which can be ameliorated, treated or cured by administration of tetrahydrobiopterin and/or a precursor thereof.

The treatment of hyperphenylalaninemia and in particular treatment of tetrahydrobiopterin-sensitive hyperphenylalaninemia requires daily administration of discrete doses dependent on the body weight of the patient. The daily dose is dispensed and administered in two portions, it may, however, also be administered in more than two portions.

### Example 1

### Dosage Parameters for a Single Administration of a Dosage Form According to the Present Invention in Humans Ranging from Infants to Adults

The following Table 1 shall illustrate the possibility of the exact dosing by using a 10% liquid formulation of tetrahydrobiopterin using a dosing pump for dispensing 500 μl aliquots (In order to administer the appropriate amount of tetrahydrobiopterin necessary for treating, ameliorating or curing tetrahydrobiopterin-sensitive hyperphenylalaninemia, the described dosage, however, has to be applied twice a day).

TABLE 1
Dosage 10 mg/kg

| Weight of patient | ml of per dose | |
|---|---|---|
| 5 kg | → 0.5 ml (1 unit) | 10% tetrahydrobiopterin solution |
| 10 kg | → 1 ml (2 units) | 10% tetrahydrobiopterin solution |
| 20 kg | → 2 ml (4 units) | 10% tetrahydrobiopterin solution |
| 40 kg | → 4 ml (8 units) | 10% tetrahydrobiopterin solution |
| 70 kg | → 7 ml (14 units) | 10% tetrahydrobiopterin solution |
| 100 kg | → 10 ml (20 units) | 10% tetrahydrobiopterin solution |

In order to reduce the number of units needed for adults of 70 kg to 100 kg and above a dosing pump capable of dispensing 1.0, 1.5 or 2.0 ml aliquots can be used alternatively.

### Example 2

### Aqueous BH₄ Compositions

Liquid Formulation A comprising 10% w/w BH₄ 2HCl

(6R)-5,6,7,8-tetrahydro-L-biopterin.2HCl 10 g

### L-cysteine HCl 5 g

sodium benzoate (EuAB) 0.2 g
demineralized water (EuAB) ad 100 ml

Liquid Formulation B comprising 10% w/w BH₄ 2HCl

(6R)-5,6,7,8-tetrahydro-L-biopterin.2HCl 10 g

### L-cysteine HCl 5 g

### Syloid® AL 5 g

sodium benzoate (EuAB) 0.2 g
demineralized water (EuAB) ad 100 ml

Liquid Formulation AB, comprising 10% w/w BH₄ 2HCl

(6R)-5,6,7,8-tetrahydro-L-biopterin.2HCl 10 g

### L-cysteine.HCl5 g

sodium benzoate (EuAB) 0,1 g
potassium sorbate (EuAB)

### Example 3

### Powder Premixtures with Tetrahydrobiopterin for the Preparation of Aqueous Solutions

The dry formulations of tetrahydrobiopterin are designed to yield 100 ml of a 10% tetrahydrobiopterin liquid formulation after reconstitution in an appropriate amount of demineralized water. The dry formulation preferably comprises a desiccant.

Solid Formulation C

(6R)-5,6,7,8-tetrahydro-L-biopterin.2HCl 10 g
L-cysteine HCl anhydrous 5 g
sodium benzoate (EuAB) 0.2 g

Solid Formulation D (with 1% Syloid®)

(6R)-5,6,7,8-tetrahydro-L-biopterin.2HCl 10 g
L-cysteine HCl anhydrous 5 g

### Syloid® AL 1 FP (5%) 0.15 g

sodium benzoate (EuAB) 0.2 g

Solid Formulation E (with 5% Syloid®)

(6R)-5,6,7,8-tetrahydro-L-biopterin.2HCl 10 g
L-cysteine HCl anhydrous 5 g

### Syloid® AL 1 FP (5%) 0.76 g

sodium benzoate (EuAB) 0.2 g

### Example 4

### Stability in Dependence on the Added Sulfhydryl Compound

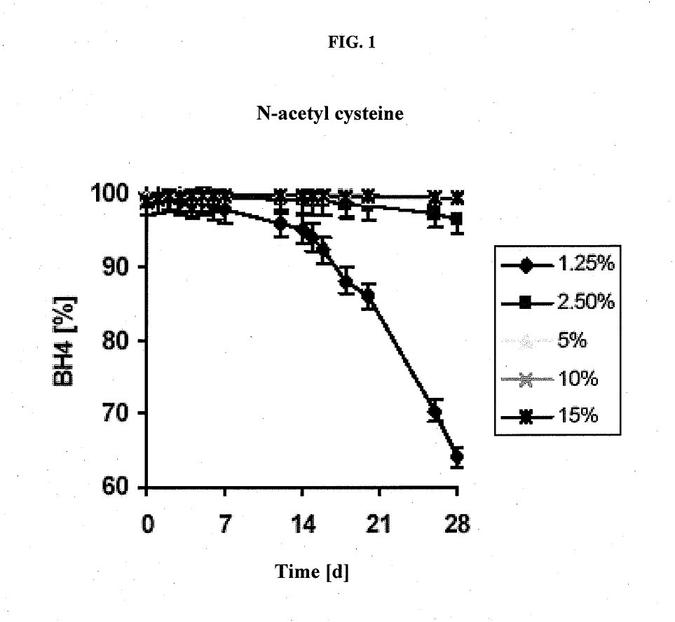

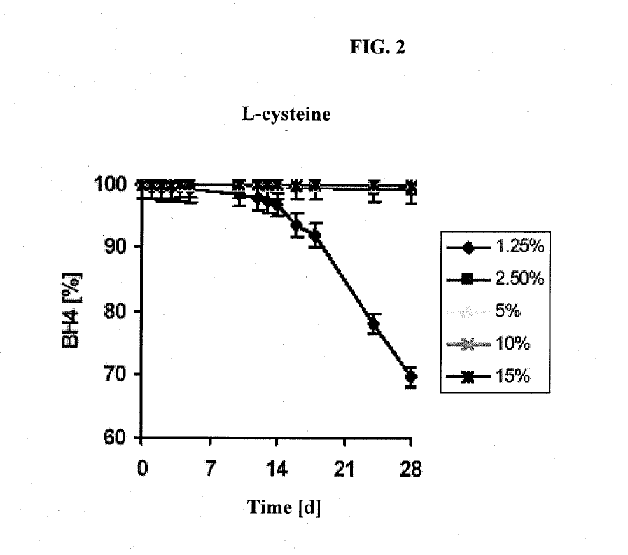

The influence of N-acetyl cysteine (FIG. 1) and L-cysteine (FIG. 2) in various concentrations on the stability of a 10% (w/w) tetrahydrobiopterin solution was evaluated. Liquid formulations were stored at room temperature, and aliquots were sampled at predetermined time points and analysed for tetrahydrobiopterin content according to the differentiated oxidation method described by Fukushima et al. (Fukushima T., Nixon J. C. —Analysis of reduced forms of biopterin in biological tissues and fluids. —Anal Biochem., 102 (1), 176-188, 1980; Fukushima T., Nixon J. C. —Chromatographic analysis of pteridines. —Methods Enzymol., 66, 429-436, 1980). Results of this study are shown in FIGS. 1 and 2.

### Example 5

### Long-Term Stability of BH₄ Liquid Formulations

The powder substances including BH₄ according to formulation A were transferred into brown glass bottles by Valois filled up to 100 g with Millipore Ultrapure water and sealed with the aludiscs and caps by Valois. Half of the bottles were additionally packaged in alubags. The samples were put into the climatic exposure test cabinet at accelerated conditions (40° C.±2° C. and 75%±5% relative humidity) and at long-term conditions (25° C.±2° C. at 60%±5% relative humidity) and were stored for 6 months. The BH₄ content at the beginning of the test and after the 6 months period was evaluated using the acidic and alkaline oxidation method by Fukushima et al. Table 2 indicates the relative BH₄ quantities measured after 6 months or 12 months of storage in comparison to the starting amount.

TABLE 2

| | Conditions | | | |
|---|---|---|---|---|
| | 25° C./60% r.h. | | 40° C./75% r.h. | |
| Sample | 1 | 2 | 3 | 4 |
| Substance | BH₄ liquid - | BH₄ liquid - | BH₄ liquid - | BH₄ liquid - |
| | with | without | with | without |
| | alubag | alubag | alubag | alubag |
| relative BH₄ | 105.1 ± 0.2 | 103.1 ± 0.2 | 103.5 ± 0.1 | 100.2 ± 0.2 |
| quantity [%]: | | | | |
| after 6 months | | | | |
| relative BH₄ | 105.0 ± 0.2 | 102.9 ± 0.2 | n.d. | n.d. |
| quantity [%]: | | | | |
| after 12 months | | | | |

n.d.: not determined

It is evident that the BH₄ content of the liquid BH₄ formulation does not deteriorate even after 6 months under accelerated storage conditions.

What is claimed is:

1. A pharmaceutical dosage form, comprising tetrahydrobiopterin, or a metabolic precursor thereof, characterized in that it comprises an aqueous solution of tetrahydrobiopterin, or a metabolic precursor thereof, and an antioxidant in a dispensing container and a dosing means for the dosed dispensation of the aqueous solution.

2. A pharmaceutical dosage form according to claim 1, characterized in that the antioxidant is a sulfhydryl compound.

3. A pharmaceutical dosage form according to claim 2, characterized in that the sulfhydryl compound is a cysteine compound, in particular selected from the group cysteine, and compounds derived therefrom, in particular selected from the group of N-acetyl cysteine, homocysteine, N-acetyl homocysteine, cysteine methyl ester, cysteine ethyl ester, homocysteine methyl ester or homocysteine ethyl ester; or mixtures of two or several different sulfhydryl compounds, optionally in the form of a salt, in particular in the form of a hydrochloride salt.

4. A pharmaceutical dosage form according any of the claims 1 to 3, characterized in that the tetrahydrobiopterin, or a metabolic precursor thereof, is present in a concentration in the range of 0.5 wt % to 50 wt %, in particular 2 wt % to 20 wt %, in particular 5 wt % to 15 wt %.

5. A pharmaceutical dosage form according any of the claims 1 to 4, characterized in that the molar ratio of the tetrahydrobiopterin, or the metabolic precursor thereof, and the antioxidant comprises a ratio of 1.5:1 to 1:4, in particular of 1:1 to 1:1.5.

6. A pharmaceutical dosage form according any of the claims 1 to 5, characterized in that the pH of the aqueous solution is below 5, in particular below 2.

7. A pharmaceutical dosage form according any of the claims 1 to 6, characterized in that the dosing means provides for the dispensation of aliquots of the aqueous solution in a range of 100 μl to 2000 μl.

8. A pharmaceutical dosage form according any of the claims 1 to 7, wherein the dosing means is firmly connected with the dispensing container.

9. A pharmaceutical dosage form according any of the claims 1 to 8, characterized in that it comprises a kit, in which the aqueous solution in a dispensing container and the dosing means are packaged separated from each other, but together in one package.

10. The use of an antioxidant, in particular a sulfhydryl compound, in particular a cysteine, for stabilizing an aqueous solution of tetrahydrobiopterin, or a metabolic precursor thereof, for a storage period of at least one month, in particular of 2, 3, 4, 5, 6, 9 or 12 months, wherein in particular the molar ratio between the tetrahydrobiopterin, or the metabolic precursor thereof, and the antioxidant comprises a ratio of about 1.5:1 to 1:4, in particular of 1:1 to 1:1.5.

11. The use according to claim 10, characterized in that the tetrahydrobiopterin, or the metabolic precursor thereof, are stable at 40° C.±2° C. and 75%±5% relative humidity and/or at 25° C.±2° C. at 60%±5% relative humidity, in particular wherein 95%, in particular 96%, 97%, 97.5%, 98% or 99% of the initial concentration of the tetrahydrobiopterin, or a metabolic precursor thereof, will be maintained upon storage of at least one month, in particular 2, 3, 4, 5, 6, 9 or 12 months.

12. The use of a pharmaceutical dosage form according to any of the claims 1 to 11 for the preparation of a medicament for treating phenylketonuria variants caused by the deficiency of tetrahydrobiopterin, in particular hyperphenylalaninemia, by means of administration of tetrahydrobiopterin, or a metabolic precursor thereof, to children or infants, in particular by administration in combination with milk or baby food.
